(19) United States
(12) Patent Application Publication
(10) Pub. No.: US 2006/0013877 A1
(43) Pub. Date: Jan. 19, 2006

(54) Hybrid of itraconazole, cyclosporine or carvedilol with a layered silicate and a process for preparing the same

(75) Inventors: Taeun Park, Seoul (KR); Hyun Jung, Seoul (KR); Hyun Mi Kim, Seoul (KR); Jin Ho Choy, Seoul (KR); Chi Won Lee, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: Nanohybrid Co., Ltd., Seoul (KR)

(21) Appl. No.: 10/522,230

(22) PCT Filed: Jul. 22, 2003
(86) PCT No.: PCT/KR03/01449

§ 371 Date: Jan. 24, 2005

(30) Foreign Application Priority Data
Jul. 22, 2002 (KR) 10-2002-0042868

(51) Int. Cl. A61K 9/14; A61K 38/13; A61K 31/496; A61K 31/403

(52) U.S. Cl. 424/484; 514/11; 514/254.07; 514/411

(57) ABSTRACT

The present invention provides the hybrids wherein itraconazole, cyclosporine or carvedilol are intercalated into the interlayers and/or absorbed onto the surfaces of layered silicates. The hybrids according to the present invention enable the maximization of bioavailability of drugs by dissolving the drugs from the layered silicates. Furthermore, the present invention provides the preparing methods of the hybrids. The process comprises: (1) dispersing a layered silicate in an aqueous solution to form an aqueous solution containing the layered silicate; (2) dissolving a drug in a organic solvent to form an organic solution containing the drug, the organic solvent having a solubility higher than that in the aqueous solution; and (3) mixing and hybridizing in the inerrface of the aqueous solution containing the layered silicate and the organic phase solution containing the drug in order to intercalate the drug into the interlayers of the layered silicate and/or to absorb the drug onto the surface of the layered silicate.

### CROSS REFERENCE TO RELATED APPLICATION

This application is a 35 U.S.C. § 371 National Phase Entry Application from PCT/KR03/01449, filed Jul. 22, 2003, and designating the U.S.

### BACKGROUND OF THE INVENTION

1. Field of the Invention

The present invention relates to hybrids of itraconazole, cyclosporine or carvedilol with layered silicate; and the production method thereof. More specifically, the present invention relates to hybrids of itraconazole, cyclosporine or carvedilol with layered silicate having good water solubility and bioavailability, and the production method thereof.

2. Description of the Related Art

Itraconazole has been well known as one of antifungal agents and is a tricyclic azole compound having the formula below (see U.S. Pat. No. 3,717,655). The chemical formula is C₃₅H₃₈Cl₂N₈O₄ and named as (±)-cis-4-[4-[4-[4-[[2-(2,4-dichlorophenyl)-2-(1H-1,2,4-triazole-1-ylmethyl)-1,3-dioxora n-4-yl]methoxy]phenyl]-1-piperazinyl]phenyl]-2,4-dihydro-2-(1-methylpropyl)-3H-1,2,4-triazole-3-one.

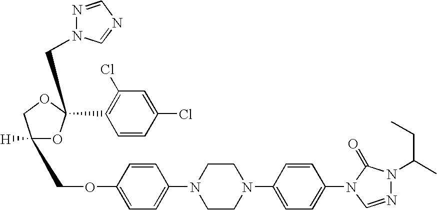

Itraconazole shows better antifungal effect than any other compounds owing to its long elimination time in the body and high permeation into proteins and lipids; however, its solubility is pH-dependent, that is, its solubility is high in acidic conditions, but low in neutral aqueous solutions. Therefore, in spite of outstanding pharmaceutical effects, itraconazole is hard to make into formulations because of the poor solubility in aqueous solutions and consequent low bioavailability.

Cyclosporine is a polymeric peptide drug that consists of 11 amino acids (a molecular weight: 1202) and is classified as cyclosporines A, B, C, D, G and the like based upon the structure, while cyclosporine A with the structure below (chemical formula C₆₂H₁₁₁N₁₁O₁₂) has been widely used for its pharmaceutical activity. Cyclosporine has been mainly used for the purpose of suppressing immune reactions after transplantation of organs and tissues although it has been also applied for inflammatory diseases such as rheumatoid arthritis.

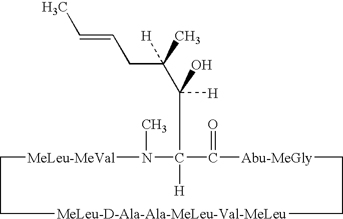

Cyclosporine has a cyclic symmetryic structure with 7 out of 11 amino acids N-methylated. Such a cyclic symmetryic structure results in very low polarity, leading to extremely low water solubility of this drug (0.04 mg/Ml H₂O, 25° C.). The extremely poor solubility of cyclosporine causes low bioavailability (approximately 30%) and it is reported that such broad deviations of the bioavailability exist among individuals as much as 5-50%. Therefore, various efforts have been made to develop improved pharmaceutical formulations for cyclosporine, focusing on the development of a method to enhance the solubility of cyclosporine.

Carvedilol is named as (±)-1-(9H-carbazole-4-yloxy)-3-[(2-(2-methoxy phenoxy)-ethyl)-amino]-2-propanol with the chemical formula of C₂₄H₂₆N₂O₄, molecular weight of 406.48 and the structure below (see U.S. Pat. No. 4,503,067)

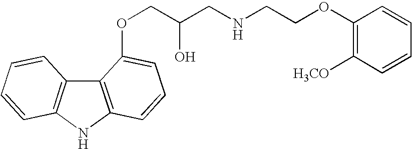

This compound is a novel drug with multiple actions, useful in treating mild to moderate hypertension. Carvedilol is known as a vasodilator and a competitive non-selective β-adrenaline receptor antagonist. The function of carvedilol as a vasodilator results from blockade of α1-adrenaline receptor and the blocking activity of β-adrenaline receptor by carvedilol leads to prevention of reflective tachycardia when the compound is used for treatment of hypertension. Such multiple actions of carvedilol are based upon the efficacy of the drug as an anti-hypertension agent. In addition, carvedilol is useful in protecting organs, especially protection of heart because of its anti-oxidative functions in reducing free radical-initiated lipid peroxidation. In addition, carvedilol is useful in treating congestive heart failure. However, carvedilol has the strong pH-dependent solubility profile, featuring especially poor solubility in the intestinal juice.

The prior conventional methods for enhancing the water-solubility in order to solve the problems of itraconazole, cyclosporine and carvedilol are divided into two categories. One is to enhance the solubility in aqueous solutions by forming such poorly soluble drugs into liposome, micro-emulsion or emulsion by using surfactants and solvents with good solubility for said drugs, as dispersants. The other is to dissolve the poorly soluble drugs in organic solvents together with hydrophilic polymers or monomeric compounds which facilitate solving the drugs in the aqueous solutions; or to mix them at high temperature into solid solutions of which the water solubility is high.

In the case of itraconazole, Janssen, the original developer of the capsule formulation, Sporanox®, used a method similar to the latter in the above to make a formulation of itraconazole. The only difference lies in that the solubility of itraconazole is enhanced by coating the surfaces of sugar beads of 600-700 μm diameters primarily with a hybrid of hydrophilic polymer hydroxypropyl methyl cellulose and itraconazole, and secondarily with polyethylene glycol over the first coating. See WO 94/05263 for details. A similar method is disclosed in Korean Patent No. 1999-001565 wherein itraconazole is solubilized by melting citric acid instead of the hydrophilic polymer at 160° C. or dissolving it in the mixed solvent of chlorinated methanol and ethanol in an amount equal to that of itraconazole and then distill the solution under reduced pressure to form a co-melted mixture, and adding appropriate excipients into said co-melted mixture. In addition to these, examples in the first category of the aforementioned methods include a method of solubility enhancing formulation for itraconazole using liposome as disclosed in WO 93/15719. In the method disclosed in said publication, itraconazole is solublilized by using phospholipid lecithin as a surfactant, and tetraglycol and dimethyl isosorbid as solvents to form single double-layered liposomes containing itraconazole.

Like itraconazole, cyclosporine employs a method fundamentally similar to the above but only with different solubilization process depending on the characteristics of each drug, or the types and the amount of solvents or additives therefor. Korean Laid-open Patent Publication No. 1998-0008239 discloses a method for solubilizing cyclosporine by using cyclic methyl ethylene carbonate or poloxamer 123 as a co-surfactant, vegetable oil (such as corn oil, sesame oil and the like) as oil and a surfactant with HLB (hydrophilic-lipophilic balance) of at least 10. Said composition is designed to solve the problem of low absorption in the body and delivery of cyclosporine by way of forming micro-emulsions in which the size of micelle can be controlled to be less than 100 nm.

Solubilization technique of carvedilol has been mainly directed to control the dissolution rate of the drug by using solid solution like cyclosporine or itraconazole. For example, Korean Patent Publication No. 2003-0019339 discloses synthesis of solid solution by mixing carvedilol and hydrophilic polymer polyethylene glycol at 70° C., and maintenance of said solid in an amorphous state so as to achieve better bioavailability than crystalline carvedilol. Another Korean Patent Publication No. 2000-0006503 aims to obtain amorphous carvedilol by synthesizing solid solution that is formed by addition of oil or fatty acid to said hydrophilic polymers.

Most of the prior arts reviewed in the above have a feature of enhancing absorption of itraconazole, cyclosporine and carvedilol with temporary super-saturation or maximized solubility thereof in gastrointestinal tracts by utilizing polymers and surfactants. However, said techniques have shortcomings that pH of the solubilized drugs increase as the drugs pass through the gastrointestinal tracts in the state of super-saturation, resulting in re-crystallization of said drugs and that such drugs can be absorbed only within a short period of time. Especially, cyclosporine solubilized in a form of emulsion has its maximum solubility instantaneously following the administration and thus it is hard to control the dissolution rate for the optimal absorption in the gastrointestinal tracts. Therefore, the need still remains to develop a more effective drug delivery system so as to deliver said drugs in the body.

### SUMMARY OF THE INVENTION

The present invention provides unique hybrids of drugs having poor water solubility such as itraconazole, cyclosporine and carvedilol with layered silicate, which enhance low solubility of these drugs since the drugs are in the amorphous state in the hybrid and result in various solubility and dissolution patterns. Since, from the point of thermodynamics, compounds or drugs are more stable in crystalline than in an amorphous form, the solubility of compounds or drugs is usually higher in the amorphous state than in the crystalline state. Considering such theoretical background, the present invention is aimed to elicit a technique to maintain the amorphous state of the hybrids produced with layered silicates and drugs such as itraconazole, cyclosporine and carvedilol.

In preferable embodiments of the present invention, said layered silicate is selected from a group of montmorillonite, beidellite and hectorite.

In addition, the present invention provides an appropriate preparing process of said hybrids.

More specifically, the present invention provides a preparing process of hybrids, comprising steps wherein drugs are dissolved in organic solvents having higher solubility than water and are intercalated into the interlayer of layered silicates and/or absorbed onto the surfaces of the layered silicates through interfacial hybridization by mixing and stirring of the above solution of drugs and the aqueous solution containing the layered silicates.

### BRIEF DESCRIPTION OF THE DRAWINGS

The above and other features and advantages of the present invention will become more apparent by describing in detail exemplary embodiments thereof with reference to the attached drawings in which:

FIG. 1 shows results of X-ray diffraction data of the hybrids of itraconazole with montmorillonite;

FIG. 2 shows results of X-ray diffraction data of the hybrids of itraconazole with hectorite;

FIG. 3 shows the solubility changes with sonication time for the commercial itraconazole formulation, Sporanox® and the hybrids according to the present invention;

FIG. 4 shows concentration change of itraconazole in the blood representing the bio-absorption characteristic of an itraconazole formulation;

FIG. 5 shows results of X-ray diffraction data of the hybrids of itraconazole with magnesium aluminum silicate;

FIG. 6 shows dissolution rate of itraconazole in the pH 1.2 solution for the hybrids of itraconazole with magnesium aluminum silicate;

FIG. 7 shows results of X-ray diffraction data of the hybrids of itraconazole with magnesium aluminum silicate having Eudragit E 1000® additionally added;

FIG. 8 shows dissolution rate of itraconazole in the pH 1.2 solution for the hybrids of itraconazole with magnesium aluminum silicate; the hybrids of itraconazole with magnesium aluminum silicate having additional Eudragit E 100®; the hybrids of itraconazole with magnesium aluminum silicate having additional Eudragit E 100® and hydroxypropyl methyl cellulose (HPMC); and Sporanox

FIG. 9 shows results of X-ray diffraction data of the hybrids of cyclosporine with montmorillonite; and

FIG. 10 shows results of X-ray diffraction data of the hybrids of carvedilol with montmorillonite.

### DETAILED DESCRIPTION OF THE INVENTION

Inventors of the present application have found that various dissolution patterns of itraconazole, cyclosporine or carvedilol can be achieved by using hybrids of with layered silicates of said drug and that bioavailability of said drugs can be maximized by sustained release of said drug from layered silicates under a condition of gastric juice and subsequently delaying recrystallization of said drug under a condition of intestinal juice having higher pH than the gastric juice.

The hybrids according to the present invention employ layered silicates as a carrier for a drug. Hereinafter, more features of silicates are provided for better understanding, but not intended to limit the scope of the present invention therein. A structural basis of layered silicates is a pyramid form of SiO₄ tetrahedron, in a layered alumino-silicates, SiO₄ tetrahedron are arranged in a way that two horizontal sheets of SiO₄ tetrahedron have apexes of tetrahedrons facing each other and connected by a metal ion (for example, aluminum) so as to form layers of a sandwich structure (for example, Si—Al—Si) aligned perpendicularly one another. Such layered structure enables the ion exchange because SiO₄ tetrahedron, the basis of each layer, can have negative charge when Si⁺⁴ replaced by Al⁺³. In some cases, the negative charge results from replacement of Al³⁺ connected by Mg²⁺. To compensate such negative charges, cations of alkaline metals or alkaline earth metals (for example, Na⁺, Ca²⁺ and the like) are present in the interlayers, wherein such interlayer metal ions are easily substituted by other cations or cationic organic components compared to metal elements within the layers such as Si, Al, Mg and the like. Moreover, the interlayer cations can be substituted by organic free bases because the organic free bases can be also intercalated into interlayers after replacing interlayer cations by hydrogen ions. Layered silicates actually have simultaneous surface adsorption of cationic organic components since the charged surface of the layered silicates as stated above features adsorption reaction rather than interlayer intercalation reaction when said interlayers exposed to outside. Thus the hybridization of layered silicates with drugs consists of interlayer-intercalation and surface-adsorption, wherein the ratio between them is responsible for different characteristics in drug delivery and can be controlled to meet the required characteristics for a drug delivery. In detail, surface-absorbed part of drugs can be easily separated and used for the fast release while the interlayer intercalated part is for the sustained release as it takes more time to be separated than the former, enabling a preferable formulation to control the rate of drug delivery.

Therefore, the hybrid of itraconazole and layered silicates according to the present invention does not form a crystalline itraconazole since the increased solubility of itraconazole is essentially due to an amorphous structure of said hybrids. Said amorphous structure was confirmed by X-ray diffraction analysis showing absence of characteristic peaks for pure crystalline itraconazole. For the production of hybrid of itraconazole with layered silicates, other drying methods than spray drying can be used because crystalline itraconazole is not formed during drying step even without using spray drying due to the outstanding stability of amorphous itraconazole in the hybrid. Spray drying is used only for easy production of fine powder of the hybrid. Same results were also taken for cyclosporine and carvedilol.

Examples of layered silicates that can be used in the hybrid according to the present invention include montmorillonite, beidellite, nontronite, hectorite, saponite, illite, celadonite, glauconite and the like. Among those montmorillonite, beidellite, hectorite, saponite and illite are preferable. Said compounds are classified into each of formulae 1 to 5 as follows, wherein said formulae represent simplified composition of actually used layered silicates and are not intended to limit the compositions of layered silicates therein.
(Al₂₋ₓMgₓ)(Si₄)O₁₀[OH]₂M⁺ⁿ_{x/n} [Formula 1]

(montmorillonite)
(Al₂)(Si₄₋ₓAlₓ)O₁₀[OH]₂M⁺ⁿ_{x/n} [Formula 2]

(beidellite)
(Mg₃₋ₓLiₓ)(Si₄)O₁₀[OH]₂M⁺ⁿ_{x/n} [Formula 3]

(hectorite)
(Mg₃₋ₓFe⁺³ₓ)(Si₄₋₂ₓAl₂ₓ)O₁₀[OH]₂M⁺ⁿ_{x/n} [Formula 4]

(saponite)
(Al_{2-x-y}Fe_{y}Mgₓ)(Si_{4-z}Al_{z})O₁₀[OH]₂M⁺ⁿ_{(x+z)/n} [Formula 5]

(illite)

In the above formulae, M stands for an interlayer metal ion, for example, alkaline metal (example: Na) or alkaline earth metal (example: Ca). x stands for the composition ratio among the interlayer metal ions, preferably from 0.1 to 0.7, more preferably from 0.2 to 0.6 and most preferably 0.3 to 0.5.

As stated above, said formulae are simplified only for representative purpose, wherein the compositions of actually used layered silicates may be varied to some extents. For example, while the montmorillonite of Formula 1 has layered structure with tetrahedrons of SiO₄, the naturally occurring montmorillonite may contain substitution in the tetrahedron such that some of Si are replaced by Al and some of Al connecting tetrahedrons of SiO₄, by other cations with +3 valence(example: Fe⁺³). Such chemical composition can be shown as (Al_{2-x-y}Fe_{y}Mgₓ)(Si_{4-z}Al_{z})O₁₀[OH]₂M⁺ⁿ_{(x+z)/n}.

The present invention also provides a preparing process of hybrids of layered silicates with drugs with poor water solubility.

In general, layered silicates may be dispersed well enough in an aqueous solution and then mixed with a drug of interest to make interlayer cations replaced by said drug or to make said drug absorbed onto the surface of the layered silicates. Considering viscosity and the like, it is preferable to disperse 1 g of montmorillonite per 1 ml of water. On the other hand, provided that a single cation in the interlayer of the layered silicates is substituted with one molecule of itraconazole and that Formula 1 corresponds to the chemical composition of said montmorillonite, the amount of itraconazole required for 1 g of montmorillonite is approximately 0.7 g. However, considering that the drugs of the present invention have extremely low water solubility (for example, the water solubility of itraconazole is about 1 mg/ml), it is practically impossible to make the hybrids of itraconazole with layered silicates in aqueous solution since it requires thousands of liters of water to dissolve such amount of itraconazole.

The present invention thus provides a preparing process of novel hybrids to overcome said problems. The process according to the present invention comprises:

(1) dispersing a layered silicate in water to form an aqueous solution containing the layered silicate;

(2) dissolving a drug in a organic solvent to form an organic solution containing the drug, the organic solvent having higher solubility than that in aqueous solution; and

(3) mixing and hybridizing in the interface between said aqueous solution containing the layered silicate and said organic solution containing the drug in order to intercalate said drug into the interlayers of said layered silicate. The interfacial hybridization in the above step (3) corresponds to interlayer intercalation/adsorption of the drug of interest in the organic phase and the layered silicates in the aqueous phase through said interface, which is formed in between said aqueous phase containing layered silicates and said organic phase containing the drug of interest. Proceeding of the interfacial hybridization through interlayer intercalation/adsorption enables continuous supply of the drug of interest from the organic phase into the aqueous phase until the completion of interlayer intercalation/adsorption between the layered silicates and the drug in the aqueous phase where the drug of interest is dissolved in an extremely small amount. As explained, the interfacial hybridization leads to the completion of intercalation/adsorption so as to increase the contents of the drug of interest in the hybrids and also the yield of the drugs.

The present invention enables a drug of interest with no charge such as itraconazole to proceed intercalation/adsorption by substituting the interlayer cations of the layered silicates with hydrogen ions before the intercalation/adsorption of step (3) since the intercalation/adsorption does not occur between the drug of interest with no charge and the layered silicates. For example, in the case of itraconazole, montmorillonite (hereinafter, MMT) has the interlayer cation (M⁺ⁿ) and if substituted with hydrogen ion (H⁺), is transformed from MMT-M⁺ⁿ to MMT-H⁺. Such hydrogen-ionized montmorillonite, MMT-H⁺, combines with the amine group (—NH— or —N═) of itraconazole which is transformed to ammonium group (—NH₂—⁺ or —NH═⁺), resulting in the hybrid of itraconazole with MMT in the form of [MMT-H⁺-itraconazole].

The content of the layered silicates in the aqueous solution of said layered silicates is from about 0.1 to about 10 wt. % and more preferably from about 0.5 to about 3 wt. %. The pH of the solution of layered silicates ranges from about 0 to about 6 and preferably from about 1 to about 4.

The organic solvents used in preparing the above solution containing a drug of interest corresponds to those with higher solubility than that in aqueous solution for the drug of interest, and the non-aqueous solvents forming the interface with the aqueous solution. Related to the solubility of the drug of interest, the organic solvents used have preferably the solubility 10 times, more preferably 100 times and most preferably 1000 times the solubility in said aqueous solutions. Such organic solvents include methylene chloride, chloroform, octanol and the like. Among those methylene chloride and chloroform are preferable and especially methylene chloride is more preferable.

The amount of the drug in the organic solution can range within the solubility limit for said drug. Further, the amount of the drug and the amount of the layered silicates depends on the content of the drug in the hybrid. Thus, the amount of the organic solvent is such to dissolve the amount of the drug required, and a volume ratio of the aqueous solvent to the organic solvent in the interface reaction is decided therefrom.

According to the present invention the content of the drug of interest in the organic solution ranges: preferably from about 1 to about 30 wt. % and more preferably from about 3 to about 10 wt. %; the volume ratio between the aqueous solvent and the organic solvent: preferably about 1:10 to about 10:1, more preferably about 1:2 to about 5:1 and most preferably 1:1 to about 2:1.

In the hybrids of itraconazole, cyclosporine and carvedilol, with layered silicates produced according to the present invention, well developed amorphous state provides higher solubility compared to that of the crystalline form. However, surface characteristics of the hybrids leads to low wettability of the hybrids in the dissolution medium. Addition of hydrophilic polymers onto said hybrids can lead to increased wettability of the hybrids in the dissolution medium. Any hydrophilic polymers are acceptable if there is no pharmaceutical restriction. Preferably Eudragit E100® (butylmethacrylate-(2-dimethylaminoethyl)methacrylate methylmethacrylate-copolymer) or hydroxypropyl methyl cellulose (HMPC) is selected.

The hydrophilic polymers are added by dissolving said polymers in a suitable solvent (example: methylene chloride and water); and the hybrids are dispersed in the solution and dried. Added amounts of the aqueous polymers are to the extent to provide sufficient wettability to the hybrids; for example, not less than 0.5 wt. % based on the weight of drugs can be used. Drying methods may include various ones known in the art, preferably spray drying.

Hereinafter, example are provided for details of the present invention but not intended to limit the scope of the present invention therein.

Hybrid of Itraconazole with Layered Silicates

### EXAMPLE 1

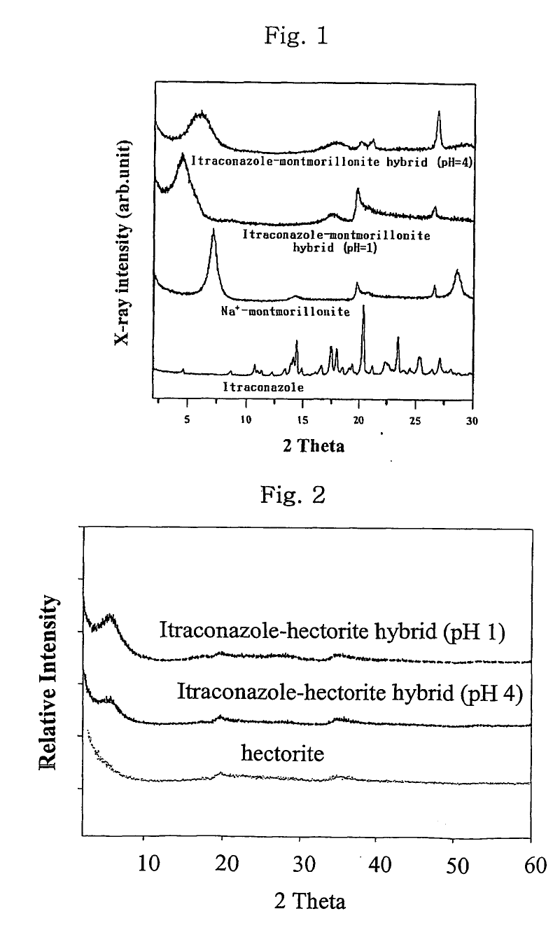

10 g of layered silicates, montmorillonite, was added into 1 liter of distilled water and stirred for 3 hours, wherein the pH was adjusted to 1 using HCl with stirring. Once equilibrium was reached at pH 1, 25 g of itraconazole was added and completely dissolved in 500 ml of methylene chloride and the solution was combined with the above aqueous solution of dispersed montmorillonite and then continuously stirred for 24 hours so as to complete the interlayer intercalation. Following the completion of the intercalation, the aqueous phase and the organic phase were separated using centrifugation, and the layered silicates precipitated in the bottom of the aqueous phase was washed with distilled water at least twice and then vacuum-dried to obtain the powder form of the hybrid of itraconazole with layered silicates. The X-ray diffraction data for the hybrids of itraconazole are shown in FIG. 1. The intercalation of itraconazole into the interlayers of the layered silicates was confirmed thereby. The content of the itraconazole in the hybrid was 55 wt. % which was calculated from the element analysis data.

### EXAMPLE 2

The hybrid was obtained employing the same conditions as those of Example 1 except using methylene chloride other than the distilled water for 3 times of washing and the content of itraconazole in the hybrid was 26 wt. % which was calculated from the element analysis data.

### EXAMPLE 3

The hybrid of itraconazole with layered silicates was obtained employing the same conditions as those of Example 1 except adjusting the pH to 4. The X-ray diffraction data for hybrids of itraconazole are shown in FIG. 1. The intercalation of itraconazole into the interlayers of the layered silicates was confirmed thereby as done in Example 1 and the content of the itraconazole in the hybrid was 55 wt. % which was calculated from the element analysis data.

### EXAMPLE 4

The hybrid of itraconazole with layered silicates was obtained employing the same conditions as those of Example 1 except using hectorite instead of montmorillonite as layered silicates. The X-ray diffraction analysis results for such itraconazole hybrid are shown in FIG. 2. The intercalation of itraconazole into the interlayers of hectorite was confirmed thereby. The content of the itraconazole in the hybrid was 16 wt. % which was calculated from the element analysis data.

### EXAMPLE 5

The hybrid of itraconazole with layered silicates was obtained employing the same conditions as those of Example 4 except adjusting the pH to 4. The X-ray diffraction analysis results for hybrids of itraconazole are shown in FIG. 2. The intercalation of itraconazole into the interlayers of the layered silicates was confirmed thereby as done in Example 3. The content of the itraconazole in the hybrid was 15 wt. % which was calculated from the element analysis data.

### EXAMPLE 6

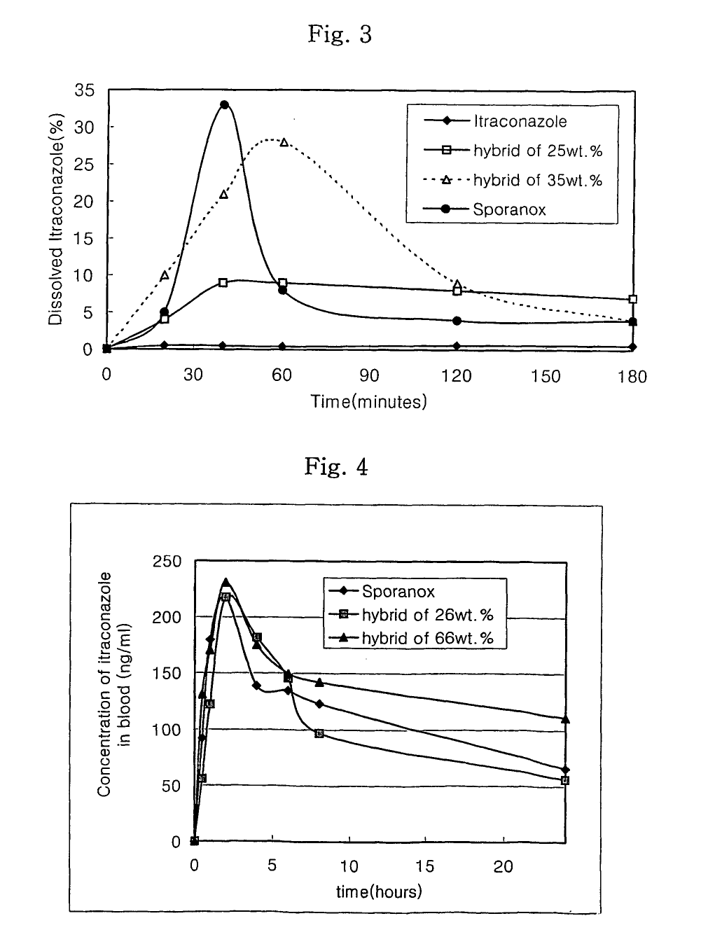

Comparison of the water solubility of itraconazole was made between the hybrids of itraconazole with layered silicates made according to the present invention, and the commercial product Sporanox® of Janssen. Sporanox and the hybrids containing 25 and 35 wt. % of itraconazole, respectively, were taken in the amounts corresponding to 100 mg of pure itraconazole; dispersed in 150 ml of the pH 1 aqueous solution; sonicated for 5 minutes; and changes of itraconazole dissolved (presented as percentage of 100 mg itraconazole) in the solution are shown in FIG. 3. The experiment was designed to measure the amount of the itraconazole in the solution which is dissolved but not recrystallized during dissolution. The hybrid with 35 wt. % of itraconazole showed the similar pattern of solubility to that of Sporanox. Furthermore, the hybrids according to the present invention sustained its solubility for a period twice as much as that for Sporanox. This implies that a period for the absorption of itraconazole in the body can be doubled in the case of the hybrid with 35 wt. % itraconazole.

The hybrid of 25 wt. % itraconazole showed a little increase in solubility but a certain level of solubility is sustained for much longer time than Sporanox.

### EXAMPLE 7

To evaluate bioequivalence of itraconazole, (A) the hybrid of 26 wt. % itraconazole from Example 2, (B) the hybrid of 66 wt. % itraconazole from Example 3, and Sporanox, were orally administered to rats in the amounts corresponding to 5 mg of pure itraconazole and blood was taken at certain times to measure the concentration of itraconazole in the plasma. Results are shown in FIG. 4. Pharmacokinetic parameters such as Tmax, Cmax and AUC are shown in Table 1. The solubility pattern for sample (A) shows a considerably low compared to the commercial itraconazole formulation, Sporanox but the actual bioavailability (presented as AUC in Table 1) reaches 90% of that for Sporanox with Tmax and Cmax similar to those for Sporanox. Sample (B) shows increased bioequivalence 20% more than that of Sporanox.

TABLE 1
Comparison among the pharmacokinetic parameters
of itraconazole formulations

| | Sporanox | Hybrid (A) | Hybrid (B) |
|---|---|---|---|
| Tₘₐₓ (hour) | 1.8 | 2.5 | 1.8 |
| Cₘₐₓ (ng/ml) | 223 | 220 | 242 |
| AUC (ng · h/ml) | 2630 | 2378 | 3327 |

### EXAMPLE 8

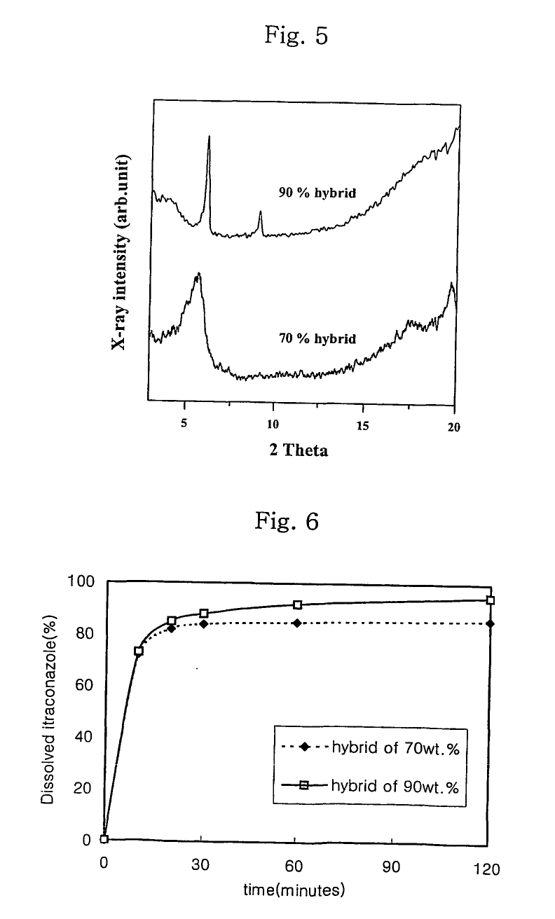

10 g of layered silicate, magnesium aluminum silicate, was added into 0.5 liter of distilled water and stirred for 3 hours, wherein the pH was adjusted to 2 using HCl with stirring. Once equilibrium was reached at pH 2, 24 g of itraconazole was added and completely dissolved in 140 ml of methylene chloride and the organic solution was combined with the above aqueous solution of dispersed magnesium aluminum silicate and then continuously stirred for 3 hours so as to complete the hybridization. Following the completion of the hybridization, the aqueous phase in the upper layer of the mixed solution was removed and slurry of hybrids in organic phase was obtained. The slurry was vacuum-dried so as to obtain the powder form of the hybrid of itraconazole with layered magnesium aluminum silicate. The X-ray diffraction data for such itraconazole hybrid are shown in FIG. 5. A specific peak for crystalline itraconazole was not found and the content of the itraconazole in the hybrid was 55 wt. % which was calculated from the element analysis data.

### EXAMPLE 9

The hybrid of itraconazole with layered magnesium aluminum silicate was obtained in the powder form under the same conditions as those of Example 8 except using 2.6 g of magnesium aluminum by removing the upper aqueous phase and vacuum-drying the lower organic phase during the hybridization. The X-ray diffraction data for such itraconazole hybrid are shown in FIG. 5. The content of the itraconazole in the hybrid was 55 wt. % which was calculated from the element analysis data.

### EXAMPLE 10

The dissolution experiments were performed using the hybrids of itraconazole with layered magnesium aluminum silicate from Examples 8 and 9. The hybrids of 70 and 90 wt. % of itraconazole, respectively, were taken in the amounts corresponding to 100 mg of pure itraconazole; dispersed in 900 ml of the pH 1.2 aqueous solution; stirred in a shaker at 200 rpm; and the concentration changes of itraconazole dissolved from each sample are shown in Table 6. The dissolution data of itraconazole from the hybrids shown in FIG. 6 confirms itraconazole of the amorphous state in the hybrid, which coincides with the result that the itraconazole exists in the amorphous state since the X-ray diffraction data from Table 6 do not show any characteristic peaks of crystalline itraconazole.

### EXAMPLE 11

10 g of the powdered hybrid of itraconazole with layered magnesium aluminum silicate ( 70 wt. % of itraconazole) from Example 8 was added to 100 ml of methylene chloride, wherein Eudragit E 100 1.4, 3.5 and 6.3 g, corresponding to 20, 50 and 90% of pure itraconazole, respectively, were dissolved; stirred for 30 minutes; and spray-dried so as to obtain the powdered hybrid of itraconazole with layered magnesium aluminum silicate coated with Eudragit E100.

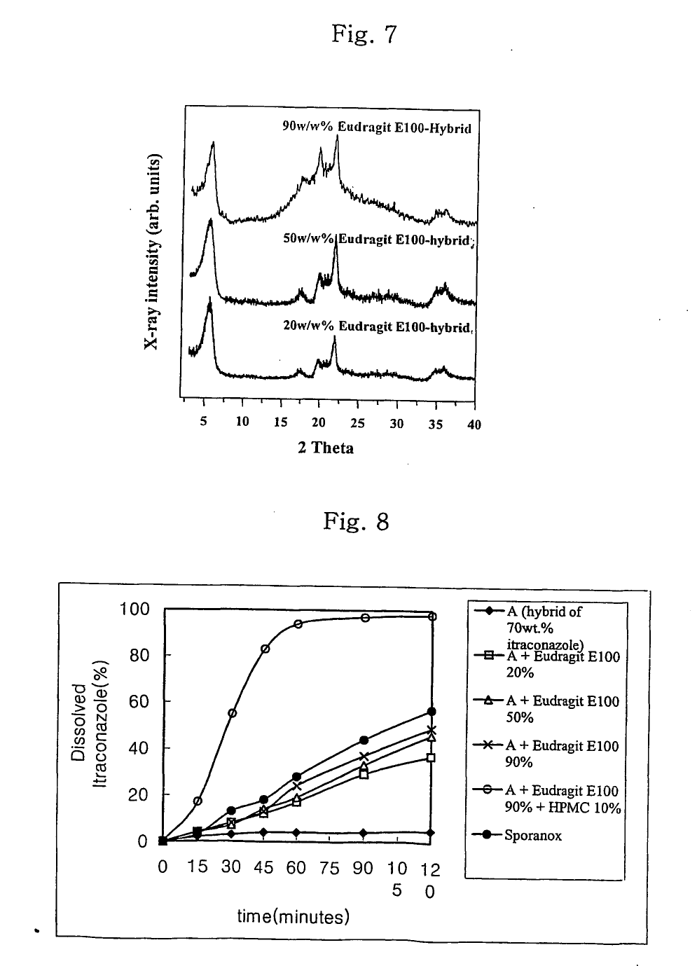

The X-ray diffraction data for such itraconazole hybrid are shown in FIG. 7. The characteristic peaks of crystalline itraconazole were not observed from these samples. The contents of the itraconazole in the hybrid were 61.4, 51.9 and 42.9 wt. %, respectively, which were calculated from the element analysis data.

### EXAMPLE 12

Among the samples from Example 11, the hybrid with the ratio 0.9 of Eudragit versus itraconazole was taken and 1 g of HPMC 606 was added to 23 g of this hybrid via wet granulation. Granules of hybrid of itraconazole with layered magnesium aluminum silicate coated with Eudragit E100 and HPMC was obtained.

### EXAMPLE 13

Comparison of the dissolution rate was made among the samples prepared without Eudragit or HPMC according to Example 8; powdered hybrids of itraconazole with layered magnesium aluminum silicate according to Example 11, wherein the ratio of Eudragit versus itraconazole was 0.2, 0.5 and 0.9, respectively; and the sample from Example 12. Each sample corresponding to 100 mg of pure itraconazole was taken and dispersed in 900 ml of the pH=1.2 aqueous solution at the dissolution test apparatus. The solution was stirred at pedal speed of 50 rpm. Aliquots of solution were taken every 15 minutes to 30 minutes to measure the amounts of dissolved itraconazole. The changes of dissolved itraconazole are shown in FIG. 8. Summarizing the results of FIGS. 3 and 6 along with those of FIG. 8 leads to the conclusion that the hybrids of itraconazole with layered silicates made according to the present invention, provide an outstanding method for various dissolution rates which can be controlled upon the dissolution conditions due to prominent stability of amorphous state of itraconazole and provide various content of itraconazole in the hybrid.

Hybrid of Cyclosporine with Layered Silicates

### EXAMPLE 14

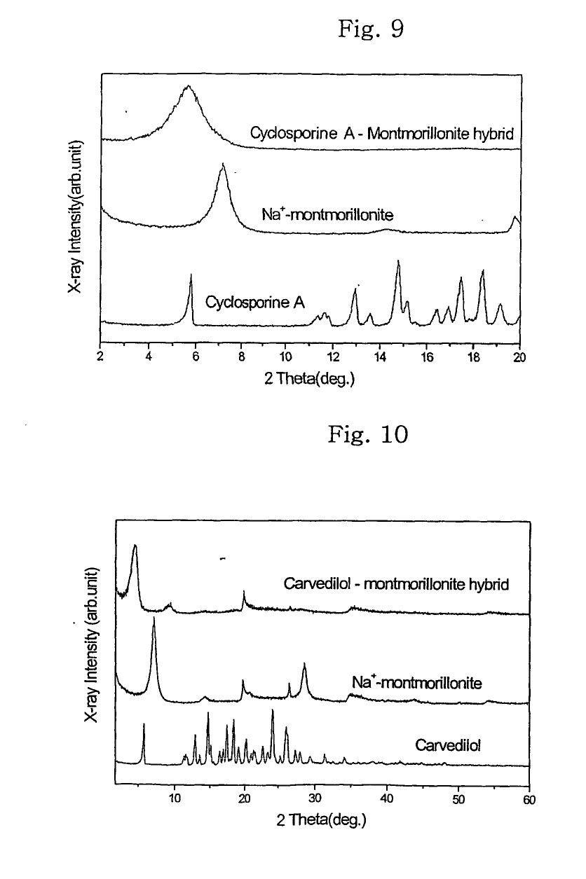

10 g of layered silicates, montmorillonite was added into 1 liter of distilled water and stirred for 3 hours, wherein the pH was adjusted to 4 using HCl with stirring. Once equilibrium was reached at pH 4, 24 g of cyclosporine was added and completely dissolved in 500 ml of methylene chloride. The organic solution was combined with the aqueous solution with dispersed montmorillonite and then continuously stirred for 24 hours so as to complete the interlayer intercalation. Following the completion of the intercalation, the aqueous phase and the organic phase were separated using centrifugation, and the precipitates in the bottom of the aqueous phase was washed with distilled water at least twice and vacuum-dried to obtain the powder form of the hybrid of cyclosporine with layered silicates. The X-ray diffraction data for the hybrid of cyclosporine are shown in FIG. 9; the intercalation of cyclosporine into the interlayers of the layered silicates was confirmed thereby; and the content of the cyclosporine in the hybrid was 50 wt. % which was calculated from the element analysis data.

Hybrid of Carvedilol with Layered Silicates

### EXAMPLE 15

10 g of montmorillonite was added into 1 liter of distilled water and stirred for 3 hours, wherein the pH was adjusted to 1 using HCl with stirring. Once equilibrium was reached at pH 1, 4 g of carvedilol was added and completely dissolved in 200 ml of methylene chloride. The organic solution was combined with the aqueous solution with dispersed montmorillonite and then continuously stirred for 24 hours so as to complete the interlayer intercalation. After the completion of the intercalation, the aqueous phase and the organic phase were separated using centrifugation, and the precipitates in the bottom of the aqueous phase was washed with distilled water at least twice and vacuum-dried to obtain the powder form of the hybrid of carvedilol with layered silicates. The X-ray diffraction data for the hybrid of carvedilol are shown in FIG. 10; the intercalation of carvedilol into the interlayers of the layered silicates was confirmed thereby; and the content of the carvedilol in the hybrid was 21 wt. % which was calculated from the element analysis data.

### EXAMPLE 16

The hybrid of carvedilol with layered silicates was made under the same conditions as those of Example 15 except for the change of pH to 2. The content of carvedilol in the hybrid was confirmed to be 25 wt. %.

### EXAMPLE 17

The hybrid of carvedilol with layered silicates was made under the same conditions as those of Example 15 except for the change of pH to 3. The content of carvedilol in the hybrid was confirmed to be 22 wt. %.

### EXAMPLE 18

The hybrid of carvedilol with layered silicates was made under the same conditions as those of Example 15 except dissolving 8.2 g of carvedilol in 200 ml of methylene chloride. The content of carvedilol in the hybrid was confirmed to be 42 wt. % which was calculated from the element analysis data.

### EXAMPLE 19

The hybrid of carvedilol with layered silicates was made under the same conditions as those of Example 18 except for the change of pH to 2. The content of carvedilol in the hybrid was confirmed to be 39 wt. %.

### EXAMPLE 20

The hybrid of carvedilol with layered silicates was made under the same conditions as those of Example 18 except for the change of pH to 3. The content of carvedilol in the hybrid was confirmed to be 38 wt. %.

### EXAMPLE 21

5g of montmorillonite was added into 1 liter of distilled water and stirred for 3 hours, wherein the pH was adjusted to 1 using HCl with stirring. Once equilibrium was reached at pH 1, 6 g of carvedilol was added and completely dissolved in 150 ml of methylene chloride. The organic solution was combined with the aqueous solution with dispersed montmorillonite and then continuously stirred for 24 hours so as to complete the intercalation. After the completion of the intercalation, the aqueous phase and the methylene chloride phase were separated using centrifugation, and the precipitates in the bottom of the aqueous phase was washed with distilled water at least twice and vacuum-dried to obtain the powder form of the hybrid of carvedilol with layered silicates. The content of the carvedilol in the hybrid was 50 wt. % which was calculated from the element analysis data.

### EXAMPLE 22

The hybrid of carvedilol with layered silicates was made under the same conditions as those of Example 21 except for the change of pH to 2. The content of carvedilol in the hybrid was confirmed to be 44 wt. %.

### EXAMPLE 23

The hybrid of carvedilol with layered silicates was made under the same conditions as those of Example 21 except for the change of pH to 3. The content of carvedilol in the hybrid was confirmed to be 47 wt. %.

### EXAMPLE 24

The hybrid of carvedilol with layered silicates was made under the same conditions as those of Example 21 except for the change of pH to 4. The content of carvedilol in the hybrid was confirmed to be 42 wt. %.

### EXAMPLE 25

The hybrid of carvedilol with layered silicates was made under the same conditions as those of Example 21 except for the change of pH to 5. The content of carvedilol in the hybrid was confirmed to be 37 wt. %.

### EXAMPLE 26

10 g of layered silicate, montmorillonite, was added into 1 liter of distilled water and stirred for 3 hours, wherein the pH was adjusted to 1 using HCl with stirring. Once equilibrium was reached with the pH 1, 12 g of carvedilol was added and completely dissolved in 300 ml of methylene chloride. The organic solution was combined with the above aqueous solution with dispersed montmorillonite and then continuously stirred for 24 hours so as to complete the intercalation. After the completion of the intercalation, the aqueous phase and the methylene chloride phase were separated using centrifugation, and the precipitates in the bottom of the aqueous phase was washed with distilled water at least twice and vacuum-dried to obtain the powder form of the hybrid of carvedilol and layered silicates. The content of the carvedilol in the hybrid was 58 wt. % which was calculated from the element analysis data.

According to the present invention, the hybrids of itraconazole, cyclosporine and carvedilol with layered silicates enable to form the stable amorphous state by said drugs, wherein such amorphous state especially provides the stability and the consequent characteristics of various solubility for each drug so as to provide an outstanding method for enhanced solubility of said drugs compared to conventional methods.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A hybrid of a drug with a layered silicate, the drug being selected from a group consisting of itraconazole, cyclosporine and carvedilol, where the drug is intercalated between the layers of the layered silicate and/or adsorbed onto the surface of the layered silicate.

2. A hybrid as defined in claim 1, wherein the drug is itraconazole.

3. A hybrid as defined in claim 1, wherein the layered silicate is selected from montmorillonite, beidellite, nontronite, hectorite, saponite, illite, celadonite and glauconite.

4. A hybrid as defined in claim 1, wherein the layered silicate is selected from montmorillonite, beidellite, saponite, hectorite and illite.

5. A process for preparing the hybrid as defined in claim 1, comprising:
(1) dispersing a layered silicate in an aqueous solution to form an aqueous solution containing the layered silicate;
(2) dissolving a drug in a organic solvent to form an organic solution containing the drug, the organic solvent having a solubility higher than that in said aqueous solution and forming an interface with said aqueous solution; and
(3) mixing and hybridizing in the interface of said aqueous solution containing the layered silicate and said organic solution containing the drug to intercalate said drug into the interlayers of said layered silicate,
wherein the drug is selected from a group consisting of itraconazole, cyclosporine and carvedilol.

6. A process as defined in claim 5, wherein a solubility of the drug in the organic solvent is at least 10 times higher than that in the aqueous solution.

7. A process as defined in claim 5, wherein the interfacial reaction is processed under an acidic condition.

8. A process as defined in claim 5, wherein pH of the aqueous solution containing the layered silicate in step (1) is between about 0 and about 6.

9. A process as defined in claim 8, wherein pH of the aqueous solution containing the layered silicate in step (1) is between about 1 and about 4.

10. A process as defined in claim 5, wherein a content of the layered silicate in the aqueous solution in step (1) is between about 1% and about 10% by weight.

11. A process as defined in claim 5, wherein a content of the 3% by weight.

12. A process as defined in claim 5, wherein a content of the drug in the organic solution in step (2) is between about 1% and about 30% by weight.

13. A process as defined in claim 5, wherein an amount of the organic solvent is such that a concentration of the layered silicate in the aqueous solution is 30% or less, and an amount of the drug in the organic solvent is 900% or less than that of the layered silicate.

14. A hybrid obtained by mixing Eudragit E100® dissolved in an organic solvent with the hybrid of itraconazole with the layered silicate as defined in claims 1, the amount of said Eudragit E100® being at least 10 by weight based on the weight of itraconazole.

15. A hybrid obtained by mixing a aqueous solution of hydroxypropyl methyl cellulose (HPMC) with the hybrid as defined in claim 14, the amount of said HPMC being is at least 0.5 by weight based on the weight of itraconazole.
